(19) United States
(12) Patent Application Publication
(10) Pub. No.: US 2026/0007383 A1
(43) Pub. Date: Jan. 8, 2026

(54) BIOLOGICAL SOUND SENSOR

(71) Applicant: Murata Manufacturing Co., Ltd., Nagaokakayo-shi (JP)

(72) Inventors: Kohei SUGAHARA, Nagaokakayo-shi (JP); Hirofumi WATANABE, Nagaokakayo-shi (JP); Hiroyuki KOMATSU, Nagaokakayo-shi (JP); Takatoshi KATO, Nagaokakayo-shi (JP)

(21) Appl. No.: 19/327,090

(22) Filed: Sep. 12, 2025

Related U.S. Application Data
(63) Continuation of application No. PCT/JP2024/002120, filed on Jan. 24, 2024.

(30) Foreign Application Priority Data
Mar. 22, 2023 (JP) 2023-045272

(51) Int. Cl. A61B 7/04

(52) U.S. Cl. CPC A61B 7/04; A61B 2562/0204; A61B 2562/16

(57) ABSTRACT

A biological sound sensor includes a housing, a diaphragm having a plate shape with a first plate surface and a second plate surface on opposite sides, and a piezoelectric element and a soft member. The diaphragm is vibratable along a thickness direction. The piezoelectric element is on the first plate surface of the diaphragm and is configured to detect a vibration of the diaphragm. The soft member has a contact surface that is configured to contact a biological body and a non-contact surface that is configured to be away from the biological body. The soft member is softer than the diaphragm. In some examples, the housing holds one of the first plate surface or the non-contact surface, and the second plate surface is bonded, from a center to a circumferential edge of the second plate surface, to the non-contact surface of the soft member.

### CROSS REFERENCE TO RELATED APPLICATIONS

This application is a continuation of International Application No. PCT/JP2024/002120, filed Jan. 24, 2024, which claims priority to Japanese Patent Application No. 2023-045272, filed Mar. 22, 2023, the entire contents of each of which are hereby incorporated by reference.

### TECHNICAL FIELD

The exemplary aspects of present disclosure relate to a biological sound sensor.

### BACKGROUND

For example, Japanese Patent No. 5, 467, 265 discloses, as an example of a biological sound sensor, a body-conducted sound sensor including a microphone element, an elastic polymer material having a sound wave input surface that is brought into contact with a human body surface to receive input of a sound wave, and a container that houses the microphone element and to which the elastic polymer material is attached with the sound wave input surface exposed. A sound wave is transmitted from the sound wave input surface to the microphone element via the elastic polymer material.

### SUMMARY OF THE DISCLOSURE

The container of the body-conducted sound sensor of Japanese Patent No. 5,467,265 has, however, an open end around the sound wave input surface, so that when the sound wave input surface is in contact with a human body surface, the open end is also in contact with the human body surface, and a noise may occur. Transmission of a noise to the microphone element may hinder accurate detection of a biological sound by the biological sound sensor.

In view of the foregoing, the exemplary aspects of the present disclosure provide techniques to suppress noise in a biological sound sensor to accurately detect a biological sound.

In some exemplary aspects, a biological sound sensor according to the present disclosure includes a housing, and a diaphragm having a plate shape with a first plate surface and a second plate surface on opposite sides. The diaphragm is vibratable along a thickness direction. The biological sound sensor also includes a piezoelectric element and a soft member. The piezoelectric element is disposed on the first plate surface of the diaphragm and is configured to detect a vibration of the diaphragm. The soft member has a contact surface that is configured to be in contact with a biological body and a non-contact surface that is configured to be away from the biological body when the contact surface is in contact with the biological body. The soft member is softer than the diaphragm. According to an exemplary aspect, the housing holds one of the first plate surface or the non-contact surface, and the second plate surface is bonded, from a center to a circumferential edge of the second plate surface, to the non-contact surface of the soft member. According to another exemplary aspect, an open end of the housing is attached to one of the first plate surface of the diaphragm or the non-contact surface of the soft member.

According to some exemplary aspects of the present disclosure, the biological sound sensor of the present disclosure is capable of suppressing a noise to accurately detect a biological sound.

### BRIEF DESCRIPTION OF DRAWINGS

FIG. 1 is a sectional view illustrating a configuration of a biological sound sensor according to a first exemplary embodiment of the present disclosure.

FIG. 2 is a sectional view illustrating a configuration of a biological sound sensor according to a modification of the first exemplary embodiment of the present disclosure.

FIG. 3 is a sectional view illustrating a configuration of a biological sound sensor according to a second exemplary embodiment of the present disclosure.

FIG. 4 is a sectional view illustrating a configuration of a biological sound sensor according to a first modification of the second exemplary embodiment of the present disclosure.

FIG. 5 is a sectional view illustrating a configuration of a biological sound sensor according to a second modification of the second exemplary embodiment of the present disclosure.

FIG. 6 is a sectional view illustrating a configuration of a biological sound sensor according to a third modification of the second exemplary embodiment of the present disclosure.

FIG. 7 is a sectional view illustrating a configuration of a biological sound sensor according to a third exemplary embodiment of the present disclosure.

FIG. 8 is a sectional view illustrating a configuration of a biological sound sensor according to another modification of the first exemplary embodiment of the present disclosure.

### DETAILED DESCRIPTION OF EMBODIMENTS

Hereinafter, embodiments will be described in detail with reference to the drawings. It is noted that the present disclosure is not limited by the embodiments. Each of the embodiments is illustrative, and it goes without saying that parts of the configurations illustrated in the different embodiments can be replaced or combined with each other.

### First Exemplary Embodiment

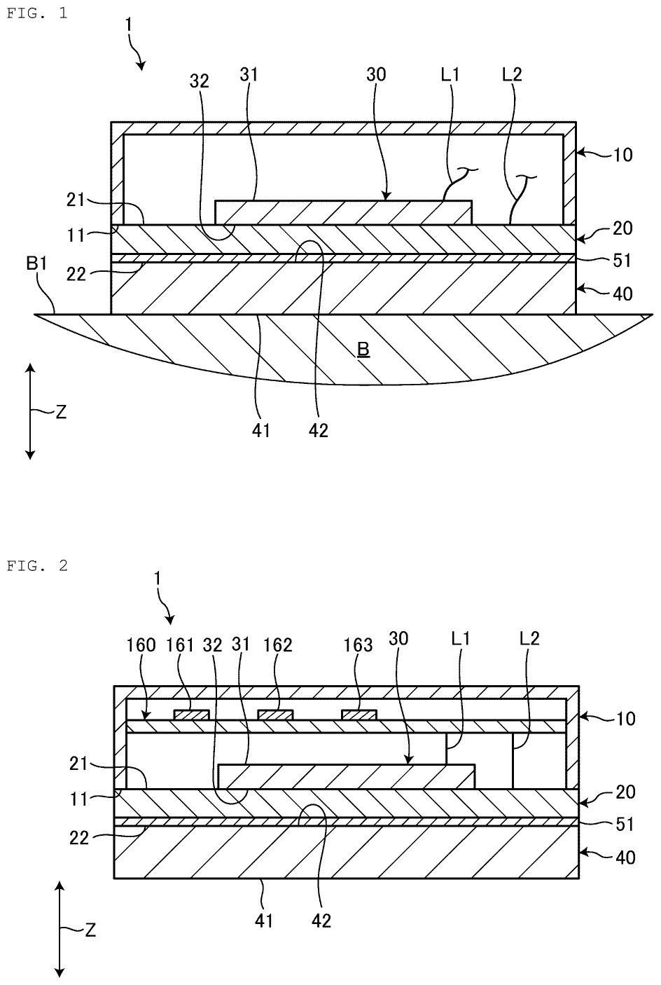

FIG. 1 is a sectional view illustrating a configuration of a biological sound sensor 1 according to a first exemplary embodiment of the present disclosure. Z direction illustrated in the drawing is a thickness direction of a diaphragm 20 to be described later. In the present specification, the phrase “plan view” can refer to a view looking at the biological sound sensor 1 along the Z direction. It is noted that FIG. 1 illustrates a state in which a soft member 40 to be described later is in contact with an outer surface B1 of a biological body B.

The biological sound sensor 1 is in contact with the outer surface B1 of the biological body B (for example, a human body) and detects a biological sound (for example, a heart sound). The biological sound sensor 1 has a columnar shape having flat surfaces on two sides in the Z direction. It goes without saying that the shape of the biological sound sensor 1 is not limited to a cylindrical shape, and may be, for example, a rectangular parallelepiped shape.

The biological sound sensor 1 includes a housing 10, a diaphragm 20, a piezoelectric element 30, and a soft member 40.

The housing 10 has a box shape of which one end in the Z direction is opened. An open end 11 of the housing 10 is annular in a plan view. The material of the housing 10 is, for example, a thermoplastic resin.

The diaphragm 20 has a plate shape having a first plate surface 21 and a second plate surface 22 on opposite sides. The diaphragm 20 has a disk shape, for example. The first plate surface 21 and the second plate surface 22 are planar. The diaphragm 20 is vibratable along the Z direction (thickness direction). The diaphragm 20 has electrical conductivity. The material of the diaphragm 20 is metal (for example, copper or nickel).

The diaphragm 20 is held by the housing 10 with the first plate surface 21 attached to the open end 11 of the housing 10. That is, the housing 10 holds the first plate surface 21. Specifically, the circumferential edge of the first plate surface 21 is held at the open end 11. Thus, the diaphragm 20 covers the opening of the housing 10. The first plate surface 21 is held at the open end 11 with an adhesive layer (not illustrated) interposed therebetween. The adhesive layer may be a tape having adhesiveness, or a cured adhesive.

The piezoelectric element 30 is housed in the housing 10. The piezoelectric element 30 detects vibration of the diaphragm 20. The piezoelectric element 30 is a piezoelectric element. The piezoelectric element 30 is, for example, a PZT-based piezoelectric ceramic of which material is lead zirconate titanate. The piezoelectric element 30 has a film shape having a first electrode surface 31 and a second electrode surface 32 on opposite sides.

The piezoelectric element 30 is disposed on the first plate surface 21 of the diaphragm 20 with the second electrode surface 32 electrically connected to the first plate surface 21. The second electrode surface 32 is disposed on the first plate surface 21 with, for example, a conductive adhesive layer (not illustrated) having electrical conductivity interposed therebetween. The conductive adhesive layer is, for example, a cured adhesive containing a conductive filler (for example, fine particles of silver). The piezoelectric element 30 deforms by the vibration of the diaphragm 20 to generate a voltage between the first electrode surface 31 and the second electrode surface 32.

The soft member 40 transmits a vibration of the biological body B to the diaphragm 20. The soft member 40 has a contact surface 41 and a non-contact surface 42. The contact surface 41 is a surface that is brought into contact with the biological body B. The non-contact surface 42 is remote from the biological body B when the contact surface 41 is in contact with the biological body B. When the contact surface 41 is separated from the biological body B, the contact surface 41 and the non-contact surface 42 have a planar shape.

In the first exemplary embodiment, the soft member 40 has a plate shape and the contact surface 41 and the non-contact surface 42 are on opposite sides. The soft member 40 has a disk shape, for example. As described above, the soft member 40 and the diaphragm 20 have plate shapes, and the piezoelectric element 30 has a film shape. Therefore, the thickness of the biological sound sensor 1 can be reduced.

The soft member 40 has flexibility. The soft member 40 is softer than the diaphragm 20. The soft member 40 has such a hardness that allows the contact surface 41, when in contact with the biological body B, to deform along the outer surface B1 of the biological body B. The Shore A hardness of the soft member 40 is 50 or less. In the first exemplary embodiment, the Asker C hardness of the soft member 40 is about 15. It goes without saying that the hardness of the soft member 40 is not limited to the above values. In addition, the soft member 40 has elasticity.

Furthermore, the soft member 40 has an acoustic impedance that is between the acoustic impedance of the skin of the biological body B and the acoustic impedance of the diaphragm 20. Thus, the soft member 40 can appropriately transmit a biological sound. The material of the soft member 40 is a polymer material (for example, silicone or urethane rubber).

The second plate surface 22 of the diaphragm 20 is bonded to the non-contact surface 42 from the center to the circumferential edge of the second plate surface 22. In other words, the second plate surface 22 is totally bonded to the non-contact surface 42. The non-contact surface 42 and the second plate surface 22 are bonded together with the first adhesive member 51 interposed therebetween. The thickness of the first adhesive member 51 is constant. No member other than the first adhesive member 51 is interposed between the non-contact surface 42 and the second plate surface 22. This suppresses the non-contact surface 42 and the contact surface 41 being shaped to protrude or be depressed in the thickness direction. The first adhesive member 51 may be a tape having adhesiveness, or may be a cured adhesive.

The housing 10 is totally located on the opposite side of the contact surface 41 across the first plate surface 21. Furthermore, in a view looking at the soft member 40 from the contact surface 41 side along the thickness direction (Z direction) of the diaphragm 20, the housing 10 is totally hidden behind the soft member 40. This suppresses the housing 10 making contact with the biological body B when the contact surface 41 is in contact with the biological body B.

The biological sound sensor 1 also includes a first electric wire L1 and a second electric wire L2. A first end of the first electric wire L1 is electrically connected to the first electrode surface 31 of the piezoelectric element 30. A first end of the second electric wire L2 is electrically connected to the first plate surface 21 of the diaphragm 20. As described above, the diaphragm 20 has electrical conductivity, and the first plate surface 21 and the second electrode surface 32 are electrically connected. Thus, the first end of the second electric wire L2 is electrically connected to the second electrode surface 32 via the diaphragm 20. A second end of the first electric wire L1 and a second end of the second electric wire L2 are outside the housing 10 and are electrically connected to, for example, a measuring instrument (not illustrated).

Next, an operation of the biological sound sensor 1 will be described. The biological sound sensor 1 of which contact surface 41 has been brought into contact with the outer surface B1 of the biological body B by a user detects a biological sound.

Specifically, vibration (hereinafter, referred to as biological vibration) of the outer surface B1 of the biological body B is transmitted from the contact surface 41 to the soft member 40. At this time as described above, the contact surface 41 is deformed along the outer surface B1 of the biological body B to be in close contact with the outer surface B1 of the biological body B. Further, as described above, the contact surface 41 being shaped to protrude or be depressed in the thickness direction is suppressed. Therefore, generation of a relatively large local stress in the soft member 40 due to the contact between the contact surface 41 and the outer surface B1 of the biological body B can be suppressed. Furthermore, generation of a noise (vibration different than biological vibration) due to the friction between the contact surface 41 and the outer surface B1 of the biological body B can be suppressed. Accordingly, the soft member 40 can accurately transmit biological vibration.

The biological vibration is transmitted to the diaphragm 20 via the soft member 40. Specifically, the biological vibration vibrates the soft member 40, and the diaphragm 20 is vibrated by the vibration of the soft member 40. Further, the piezoelectric element 30 is vibrated by the vibration of the diaphragm 20. As a result, in the piezoelectric element 30, a voltage corresponding to the vibration of the piezoelectric element 30 is generated across the first electrode surface 31 and the second electrode surface 32. The waveform of the voltage generated by the piezoelectric element 30 corresponds to the waveform of the biological sound. That is, the biological sound sensor 1 detects the voltage generated in the piezoelectric element 30 as the biological sound. The voltage generated in the piezoelectric element 30 is output to a measuring instrument via the first electric wire L1 and the second electric wire L2.

As described above, contact of the housing 10 with the biological body B is suppressed when the contact surface 41 is in contact with the biological body B. Therefore, generation of noise due to the contact of the housing 10 with the biological body B is suppressed. Accordingly, the biological sound sensor 1 can accurately detect biological sound.

### Modification of First Exemplary Embodiment

Next, a biological sound sensor 1 according to a modification of the first exemplary embodiment of the present disclosure will be described mainly with emphasis on the differences from the biological sound sensor 1 according to the first exemplary embodiment.

FIG. 2 is a sectional view illustrating a configuration of the biological sound sensor 1 according to the modification of the first exemplary embodiment of the present disclosure. The biological sound sensor 1 according to the modification further includes an electrical circuit 160 as compared with the biological sound sensor 1 of the first exemplary embodiment.

The electrical circuit 160 has a plate shape and is housed in a housing 10. Second ends of a first electric wire L1 and a second electric wire L2 are electrically connected to the electrical circuit 160. That is, a first electrode surface 31 is electrically connected to the electrical circuit 160 via the first electric wire L1, and a second electrode surface 32 is electrically connected to the electrical circuit 160 via a diaphragm 20 and the second electric wire L2. As a result, a voltage generated in a piezoelectric element 30 is output to the electrical circuit 160.

The electrical circuit 160 includes an amplifier 161, a filter unit 162, and an output unit 163. The amplifier 161 amplifies the voltage output from the piezoelectric element 30. The filter unit 162 removes electric noise generated in the electrical circuit 160. The output unit 163 outputs a voltage (voltage corresponding to a biological sound) amplified by the amplifier 161 to a measuring instrument. The output unit 163 is, for example, a connector electrically connected to the measuring instrument via an electric wire (not illustrated). It is noted that the output unit 163 may be a transmitter that wirelessly transmits a signal including information corresponding to the voltage amplified by the amplifier 161 to the measuring instrument.

The first electric wire L1 and the second electric wire L2 of the modification are housed in the housing 10, and the lengths of the first electric wire L1 and the second electric wire L2 of the modification are shorter than the lengths of the first electric wire L1 and the second electric wire L2 of the first exemplary embodiment. This enables suppressing an electric noise entering the first electric wire L1 and the second electric wire L2 from outside. Accordingly, the biological sound sensor 1 can accurately output biological sounds.

Further, the piezoelectric element 30 may include a piezoelectric body (not illustrated) that exhibits a piezoelectric effect, a first electrode (not illustrated) and a second electrode (not illustrated) that sandwich the piezoelectric body in the Z direction. By the piezoelectric body deformed by the vibration of the diaphragm 20, a voltage is generated across the first electrode and the second electrode. The first electrode has a first electrode surface 31, and the second electrode has a second electrode surface 32. In this case, the diaphragm 20 may not have electrical conductivity, and a first end of the second electric wire L2 is electrically connected to the second electrode.

### Second Exemplary Embodiment

Next, a biological sound sensor 1 according to a second exemplary embodiment of the present disclosure will be described mainly with emphasis on the differences from the biological sound sensor 1 according to the modification of the first exemplary embodiment.

FIG. 3 is a sectional view illustrating a configuration of the biological sound sensor 1 according to the second exemplary embodiment of the present disclosure. The biological sound sensor 1 according to the second exemplary embodiment does not include a first electric wire L1 and a second electric wire L2 as compared with the biological sound sensor 1 of the first exemplary embodiment.

A housing 210 of the second exemplary embodiment has a sleeve shape having openings on two sides in the Z direction. In the second exemplary embodiment, an open end of the housing 210 on the soft member 40 side is referred to as a first open end 211a, and an open end on the side opposite to the first open end 211a is referred to as a second open end 211b. The housing 210 has electrical conductivity. The material of the housing 210 may be, for example, a metal or a thermoplastic resin containing a conductive filler (for example, carbon fine particles (namely, carbon black)).

In the second exemplary embodiment, an electrical circuit 260 has a disk shape, and the second open end 211b holds the circumferential edge portion of the electrical circuit 260. Accordingly, the electrical circuit 260 covers the opening on the second open end 211b side of the housing 210. The second open end 211b holds the electrical circuit 260 with a conductive adhesive layer (not illustrated) interposed therebetween. The housing 210 and the electrical circuit 260 are thereby electrically connected to each other.

A piezoelectric element 30 and a diaphragm 20 are housed in the housing 210. A conductive adhesive layer (not illustrated) is interposed between a first electrode surface 31 and the electrical circuit 260, and the first electrode surface 31 and the electrical circuit 260 are in contact with the conductive adhesive layer to be electrically connected to each other.

In the second exemplary embodiment, a soft member 40 is held by the housing 210 with a non-contact surface 42 attached to the first open end 211a of the housing 210. That is, the housing 210 holds the non-contact surface 42. Specifically, the circumferential edge portion of the non-contact surface 42 is held by the first open end 211a. Accordingly, the soft member 40 covers the opening on the first open end 211a side of the housing 210.

A second plate surface 22 of the diaphragm 20 and the first open end 211a of the housing 210 are bonded to the non-contact surface 42 with a second adhesive member 252 having electrical conductivity interposed therebetween. The second adhesive member 252 may be, for example, a cured adhesive containing a conductive filler (for example, fine particles of silver), or may be an adhesive tape containing a conductive filler. The housing 210 is totally located on the opposite side of a contact surface 41 across the non-contact surface 42. This suppresses noise generated by the housing 210 making contact with the biological body B.

Similarly to the first exemplary embodiment, the second plate surface 22 of the diaphragm 20 is bonded to the non-contact surface 42 from the center to the circumferential edge of the second plate surface 22 with the second adhesive member 252 interposed therebetween. The thickness of the second adhesive member 252 is constant. No member other than the second adhesive member 252 is interposed between the non-contact surface 42 and the second plate surface 22. Similarly to the first exemplary embodiment, this suppresses the non-contact surface 42 and the contact surface 41 being shaped to protrude or be depressed in the thickness direction.

The second adhesive member 252 is continuous from the second plate surface 22 of the diaphragm 20 to the second open end 211b of the housing 210. That is, the diaphragm 20 and the housing 210 are electrically connected via the second adhesive member 252. Therefore, the second electrode surface 32 of the piezoelectric element 30 is electrically connected to the electrical circuit 260 via the diaphragm 20, the second adhesive member 252, and the housing 210. Accordingly, a voltage generated in the piezoelectric element 30 is output to the electrical circuit 260.

In the biological sound sensor 1 of the second exemplary embodiment, a voltage generated in the piezoelectric element 30 is output to the electrical circuit 260 without passing through an electric wire. Therefore, the thickness of the biological sound sensor 1 can be reduced.

### First Modification of Second Exemplary Embodiment

Next, a biological sound sensor 1 according to a first modification of the second exemplary embodiment of the present disclosure will be described mainly with emphasis on the differences from the biological sound sensor 1 (see FIG. 3) according to the second exemplary embodiment.

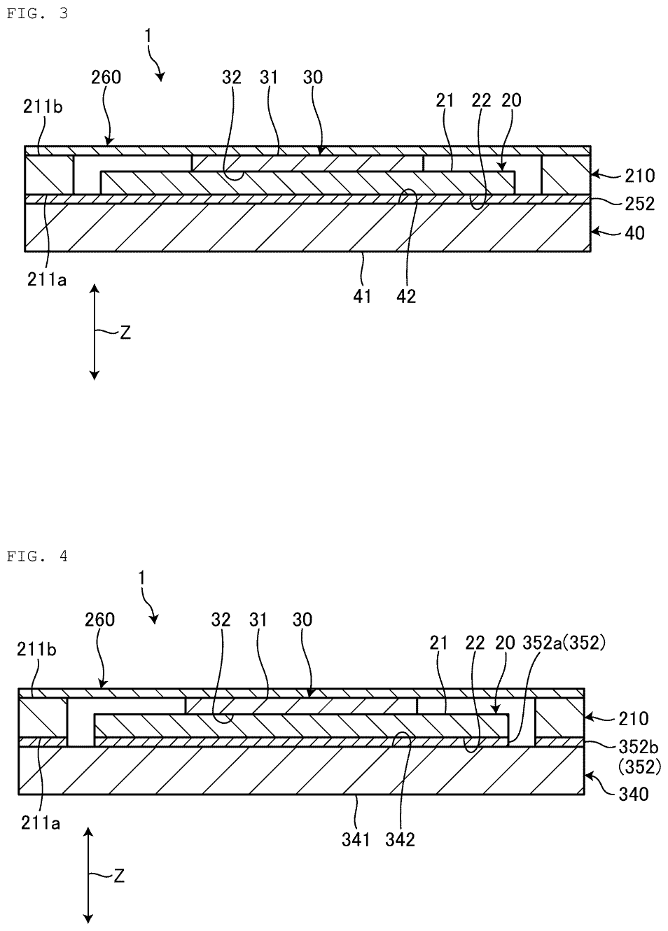

FIG. 4 is a sectional view illustrating a configuration of the biological sound sensor 1 according to the first modification of the second exemplary embodiment of the present disclosure. In the first modification, a soft member 340 has electrical conductivity. The material of the soft member 340 is, for example, a polymer material containing a conductive filler. In the first modification, the biological sound sensor 1 includes two second adhesive members 352. Hereinafter, in the first modification, one of the second adhesive members 352 is referred to as a primary second adhesive member 352a, and the other of the second adhesive members 352 is referred to as a secondary second adhesive member 352b.

A second plate surface 22 of a diaphragm 20 is bonded to a non-contact surface 342 with the primary second adhesive member 352a interposed therebetween. No member other than the primary second adhesive member 352a is interposed between the non-contact surface 342 and the second plate surface 22. A diaphragm 20 and the soft member 340 are electrically connected to each other via the primary second adhesive member 352a.

A first open end 211a of a housing 210 is bonded to the non-contact surface 342 with the secondary second adhesive member 352b interposed therebetween. The secondary second adhesive member 352b has an annular shape that surrounds the primary second adhesive member 352a in a plan view. Accordingly, the soft member 340 and the housing 210 are electrically connected to each other via the secondary second adhesive member 352b.

In the first modification, a second electrode surface 32 of a piezoelectric element 30 is electrically connected to an electrical circuit 260 via the diaphragm 20, the primary second adhesive member 352a, the soft member 340, the secondary second adhesive member 352b, and the housing 210.

In the first modification, the primary second adhesive member 352a and the secondary second adhesive member 352b may be integrated.

### Second Modification of Second Exemplary Embodiment

Next, a biological sound sensor 1 according to a second modification of the second exemplary embodiment of the present disclosure will be described mainly with emphasis on the differences from the biological sound sensor 1 (see FIG. 3) according to the second exemplary embodiment.

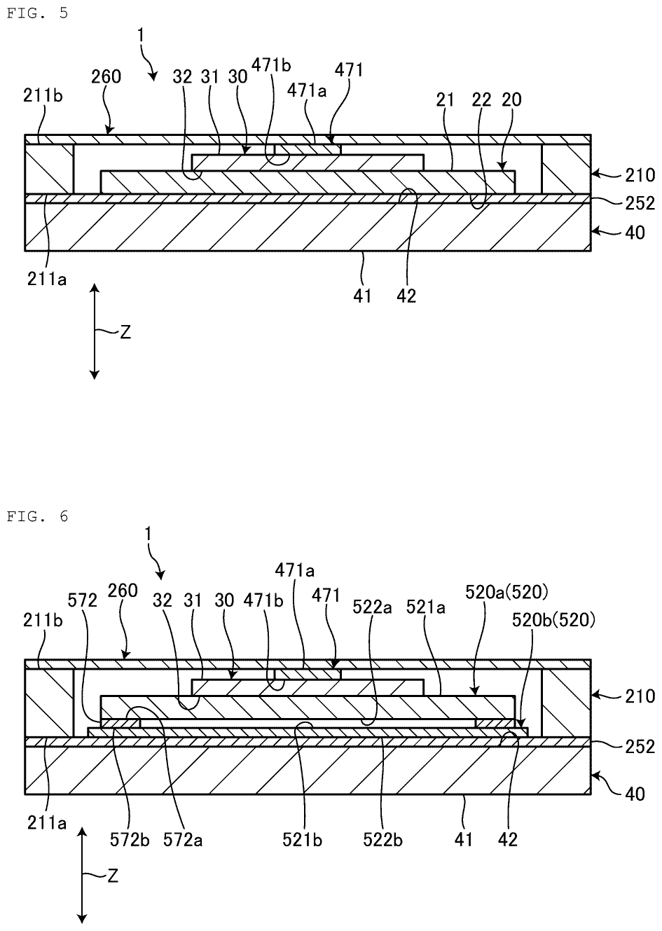

FIG. 5 is a sectional view illustrating a configuration of the biological sound sensor 1 according to the second modification of the second exemplary embodiment of the present disclosure. The biological sound sensor 1 of the second modification further includes a first coupling member 471. A first coupling member 471 is housed in a housing 210.

The first coupling member 471 has a pillar shape having a first end surface 471a and a second end surface 471b, and has electrical conductivity. The area of each of the first end surface 471a and the second end surface 471b is smaller than the area of a first electrode surface 31. The material of the first coupling member 471 is, for example, a thermoplastic resin containing a conductive filler.

The first coupling member 471 couples a piezoelectric element 30 and an electrical circuit 260 to each other with the first electrode surface 31 and the electrical circuit 260 electrically connected to each other. Specifically, the first end surface 471a and the electrical circuit 260 are electrically connected to each other via a conductive adhesive layer (not illustrated). Further, the second end surface 471b and the first electrode surface 31 are electrically connected to each other via a conductive adhesive layer (not illustrated). In a plan view, the first coupling member 471 is located at the center of the piezoelectric element 30.

In the second modification, the piezoelectric element 30 and a diaphragm 20 vibrate in a state of being supported by the first coupling member 471. That is, the piezoelectric element 30 and the diaphragm 20 vibrate with the first coupling member 471 serving as a fulcrum. This increases the amplitudes of the piezoelectric element 30 and the diaphragm 20 in response to a biological vibration. That is, the first coupling member 471 amplifies the amplitudes of the piezoelectric element 30 and the diaphragm 20. Therefore, the first coupling member 471 can improve the sensitivity of the biological sound sensor 1.

### Third Modification of Second Exemplary Embodiment

Next, a biological sound sensor 1 according to a third modification of the second exemplary embodiment of the present disclosure will be described mainly with emphasis on the differences from the biological sound sensor 1 (see FIG. 5) according to the second modification of the second exemplary embodiment.

FIG. 6 is a sectional view illustrating a configuration of the biological sound sensor 1 according to the third modification of the second exemplary embodiment of the present disclosure. The biological sound sensor 1 of the third modification includes two diaphragms 520 and further includes a second coupling member 572. Hereinafter, in the third modification, one of the diaphragms 520 is referred to as a first diaphragm 520a, and the other of the diaphragms is referred to as a second diaphragm 520b. The first diaphragm 520a and the second diaphragm 520b may differ in size, or may have the same size. A piezoelectric element 30, the first diaphragm 520a, the second diaphragm 520b, a first coupling member 471, and the second coupling member 572 are housed in a housing 210.

The first diaphragm 520a has a plate shape having a first plate surface 521a and a second plate surface 522a on opposite sides, has electrical conductivity, and is vibratable along the thickness direction. The second diaphragm 520b has a plate shape having a first plate surface 521b and a second plate surface 522b located on opposite sides, has electrical conductivity, and is vibratable along the thickness direction.

Similarly to the diaphragm 20 of the second modification of the second exemplary embodiment, the piezoelectric element 30 is disposed on the first plate surface 521a of the first diaphragm 520a. That is, the piezoelectric element 30 is disposed on the first plate surface 521a of the first diaphragm 520a with a second electrode surface 32 and the first plate surface 521a of the first diaphragm 520a electrically connected to each other, and detects vibration of the first diaphragm 520a.

The second coupling member 572 has electrical conductivity, and couples the first diaphragm 520a and the second diaphragm 520b to each other with the second plate surface 522a of the first diaphragm 520a and the first plate surface 521b of the second diaphragm 520b electrically connected to each other. The material of the second coupling member 572 is, for example, a thermoplastic resin containing a conductive filler. The second coupling member 572 has an annular shape that surrounds the first coupling member 471 in a view looking at the second diaphragm 520b along the Z direction (thickness direction). The second coupling member 572 is sandwiched between the circumferential edge portion of the first diaphragm 520a and the circumferential edge portion of the second diaphragm 520b.

The second coupling member 572 has a first end surface 572a and a second end surface 572b. The first end surface 572a and the second plate surface 522a of the first diaphragm 520a are electrically connected to each other via a conductive adhesive layer (not illustrated). The second end surface 572b and the first plate surface 521b of the second diaphragm 520b are electrically connected to each other via a conductive adhesive layer (not illustrated).

A soft member 40 is softer than the first diaphragm 520a and the second diaphragm 520b. The soft member 40 transmits a biological vibration to the second diaphragm 520b. Similarly to the second exemplary embodiment, the second plate surface 522b of the second diaphragm 520b is bonded to a non-contact surface 42 of the soft member 40 from the center to the circumferential edge of the second plate surface 522b with a second adhesive member 252 interposed therebetween. No member other than the second adhesive member 252 is interposed between the non-contact surface 42 and the second plate surface 522b of the second diaphragm 520b. Similarly to the second exemplary embodiment, this suppresses the non-contact surface 42 and the contact surface 41 being shaped to protrude or be depressed in the thickness direction. In addition, similarly to the second exemplary embodiment, a first open end 211a of the housing 210 is bonded to the non-contact surface 42 with the second adhesive member 252 interposed therebetween.

With the biological sound sensor 1 configured as described above, the second electrode surface 32 is electrically connected to an electrical circuit 260 via the first diaphragm 520a, the second coupling member 572, the second diaphragm 520b, the second adhesive member 252, and the housing 210.

Since the second coupling member 572 surrounds the first coupling member 471 in a view looking at the second diaphragm 520b along the Z direction as described above, the piezoelectric element 30 and the first diaphragm 520a vibrate in a state of being supported by the first coupling member 471 and the second coupling member 572. This increases the amplitudes of the piezoelectric element 30 and the first diaphragm 520a in response to a biological vibration. That is, the first coupling member 471 and the second coupling member 572 amplify the amplitudes of the piezoelectric element 30 and the first diaphragm 520a. Therefore, the first coupling member 471 and the second coupling member 572 can improve the sensitivity of the biological sound sensor 1.

### Third Exemplary Embodiment

Next, a biological sound sensor 1 according to a third exemplary embodiment of the present disclosure will be described mainly with emphasis on the differences from the biological sound sensor 1 (see FIG. 3) according to the second exemplary embodiment.

FIG. 7 is a sectional view illustrating a configuration of the biological sound sensor 1 according to the third exemplary embodiment of the present disclosure. The biological sound sensor 1 of the third exemplary embodiment further includes a second housing 681 and a mounting body 682.

A housing 210 is attached to the second housing 681. Specifically, the second housing 681 has a recess 681a to which an electrical circuit 260 and the housing 210 fit. The material of the second housing 681 is, for example, a thermoplastic resin.

The second housing 681 is totally located on the opposite side of a contact surface 41 across a non-contact surface 42 when the housing 210 is attached to the second housing 681. Accordingly, a noise caused by the second housing 681 making contact with a biological body B can be suppressed.

Using the mounting body 682, the second housing 681 to which the housing 210 is attached is attached to the biological body B with the contact surface 41 in contact with an outer surface B1 of the biological body B. The mounting body 682 has a form of a band and is wound around, for example, the torso and an arm of the biological body B. Accordingly, the biological sound sensor 1 can be mounted on the biological body B, and can continuously detect a biological sound. With this, the close contact between the contact surface 41 and the outer surface B1 of the biological body B can be enhanced to suppress a slide between the contact surface 41 and the outer surface B1 of the biological body B.

It is noted that the housing 210 of the biological sound sensor 1 according to the first exemplary embodiment (see FIG. 1) may be attached to the second housing 681. In this case, the second housing 681 is totally located on the opposite side of the contact surface 41 across a first plate surface 21.

### Other Modifications

It is noted that the above embodiments are for facilitating understanding of the present disclosure, and are not intended to limit the interpretation of the present disclosure. The present disclosure may be modified or improved without departing from the spirit thereof, and the present disclosure includes equivalents thereof.

For example, the electrical circuit 160 may not include one of the amplifier 161 and the filter unit 162. In a view looking at the soft member 40 from the contact surface 41 side along the thickness direction of the diaphragm 20, a part of the housings 10 and/or 210 may not be behind the soft member 40 and be visible.

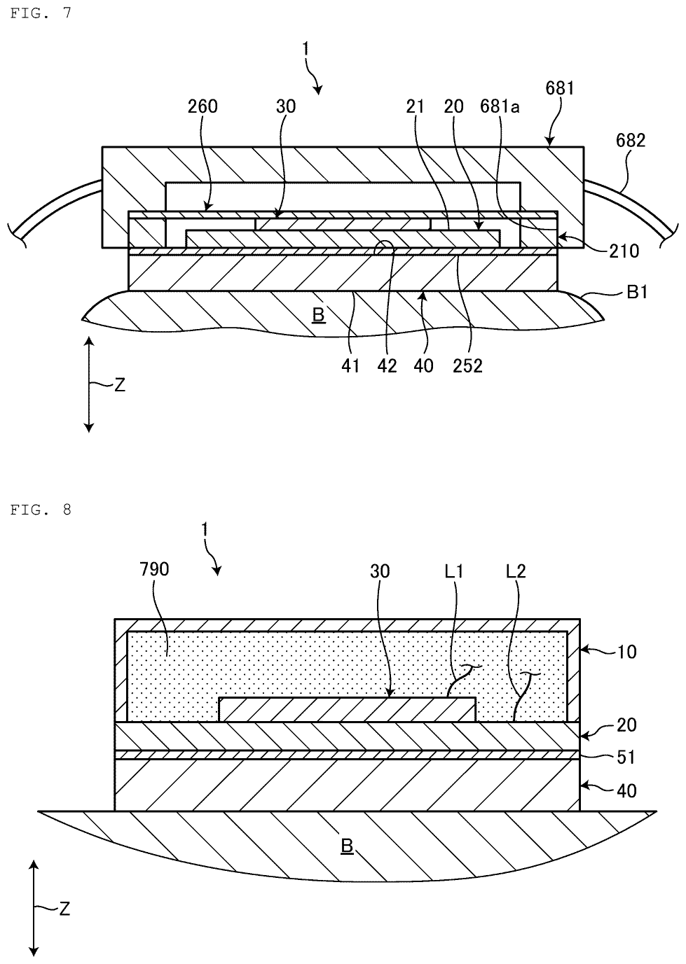

FIG. 8 is a sectional view illustrating a configuration of a biological sound sensor 1 according to another modification of the first exemplary embodiment of the present disclosure. The inside of a housing 10 of the modification is filled with a filling member 790.

The filling member 790 is softer than a soft member 40. The material of the filling member 790 is a polymer material (for example, silicone or urethane rubber).

The amplitude of a diaphragm 20 vibrated by a biological vibration depends on the hardness of the filling member 790. That is, the sensitivity of the biological sound sensor 1 can be controlled by the hardness of the filling member 790. In addition, the filling member 790 suppresses an impact that acts on a piezoelectric element 30 and the diaphragm 20 in case of dropping the biological sound sensor 1, and thus can prevent damage to the piezoelectric element 30 and the diaphragm 20. Furthermore, the filling member 790 can suppress an externally entering noise other than biological vibrations. It is noted that the filling member 790 may be filled inside the housing 210 of the biological sound sensor 1 of the second exemplary embodiment.

In the biological sound sensor 1 of each of the above-described embodiments and the modifications, similarly to the biological sound sensor 1 of the first exemplary embodiment, the contact surface 41 is in close contact with the outer surface B1 of the biological body B when the contact surface 41 is in contact with the biological body B, so that generation of a relatively large local stress in the soft member 40 can be suppressed, and generation of a noise due to the friction between the contact surface 41 and the outer surface B1 of the biological body B can be suppressed. Furthermore, in the biological sound sensor 1 of each of the embodiments and the modifications, the contact of the housings 10 and/or 210 and the second housing 681 with the biological body B is suppressed, so that generation of a noise due to the contact between the housing 10 and/or 210 and the biological body B is suppressed. Accordingly, the biological sound sensor 1 can accurately detect biological sound.

### DESCRIPTION OF REFERENCE SYMBOLS

1: Biological sound sensor
10: Housing
20: Diaphragm
21: First plate surface
22: Second plate surface
30: Piezoelectric element
31: First electrode surface
32: Second electrode surface
40: Soft member
41: Contact surface
42: Non-contact surface
51: First adhesive member (adhesive member)
160: Electrical circuit
252: Second adhesive member (adhesive member)
471: First coupling member
471a: First end surface
471b: Second end surface (end surface of first coupling member)
572: Second coupling member
681: Second housing
682: Mounting body
790: Filling member
B: Biological body
B1: Outer surface

What is claimed is:

1. A biological sound sensor comprising:
a housing;
a diaphragm having a plate shape with a first plate surface and a second plate surface on opposite sides of the diaphragm, and the diaphragm being vibratable along a thickness direction thereof;
a piezoelectric element on the first plate surface of the diaphragm, the piezoelectric element configured to detect a vibration of the diaphragm; and
a soft member having a contact surface that is configured to contact a biological body and a non-contact surface that is configured to be away from the biological body when the contact surface is in contact with the biological body, the soft member comprising a material that is softer than the diaphragm,
wherein:
the housing is configured to hold one of the first plate surface of the diaphragm and the non-contact surface of the soft member, and
the second plate surface of the diaphragm is coupled, from a center to a circumferential edge of the second plate surface, to the non-contact surface of the soft member.

2. The biological sound sensor according to claim 1, wherein the soft member has a plate shape having the contact surface and the non-contact surface on opposite sides.

3. The biological sound sensor according to claim 1, wherein the housing is totally located on an opposite side of the contact surface of the soft member across the first plate surface of the diaphragm or across the non-contact surface of the soft member.

4. The biological sound sensor according to claim 1, wherein the housing is totally located behind the soft member in a view looking at the soft member along the thickness direction of the diaphragm from the contact surface of the soft member.

5. The biological sound sensor according to claim 1, further comprising:
an electrical circuit that is held by the housing, wherein:
the diaphragm is electrically conductive,
the piezoelectric element has a first electrode surface and a second electrode surface,
the first electrode surface is electrically connected to the electrical circuit, and
the second electrode surface is on the first plate surface of the diaphragm and is electrically connected to the electrical circuit via the diaphragm.

6. The biological sound sensor according to claim 5, wherein:
the electrical circuit and the piezoelectric element are in the housing, and
the housing is configured to hold the first plate surface of the diaphragm.

7. The biological sound sensor according to claim 5, wherein:
the piezoelectric element and the diaphragm are in the housing;
the housing is electrically conductive and electrically connected to the electrical circuit,
the housing is configured to hold the non-contact surface of the soft member;
the second plate surface of the diaphragm and the housing are coupled to the non-contact surface of the soft member by an adhesive member that is electrically conductive; and
the second electrode surface of the piezoelectric element is electrically connected to the electrical circuit via the diaphragm, the adhesive member, and the housing.

8. The biological sound sensor according to claim 7, wherein:
the soft member is electrically conductive, and
the second electrode surface of the piezoelectric element is electrically connected to the electrical circuit via the diaphragm, the adhesive member, the soft member, and the housing.

9. The biological sound sensor according to claim 7, further comprising:
a first coupling member having a pillar shape, the first coupling member being electrically conductive, and electrically connected with the first electrode surface of the piezoelectric element with the electrical circuit,
wherein an area of an end surface of the first coupling member on the first electrode surface of the piezoelectric element is smaller than an area of the first electrode surface of the piezoelectric element.

10. The biological sound sensor according to claim 1, further comprising a filling member that is softer than the soft member and is inside the housing.

11. The biological sound sensor according to claim 1, further comprising:
a second housing to which the housing is attached; and
a mounting body configured to mount the second housing to the biological body.

12. The biological sound sensor according to claim 11, wherein the second housing is totally located on an opposite side of the contact surface of the soft member across the first plate surface of the diaphragm.

13. The biological sound sensor according to claim 11, wherein the second housing is totally located on an opposite side of the contact surface of the soft member across the non-contact surface of the soft member.

14. A biological sound sensor comprising:
a housing being electrically conductive;
an electrical circuit held by the housing;
a first diaphragm and a second diaphragm each having a plate shape with a first plate surface and a second plate surface on opposite sides, the first diaphragm and the second diaphragm being electrically conductive, and being vibratable along a thickness direction;
a piezoelectric element that has a first electrode surface and a second electrode surface, and is on the first plate surface of the first diaphragm, the second electrode surface of the piezoelectric element and the first plate surface of the first diaphragm being electrically connected to each other, and the piezoelectric element being configured to detect a vibration of the first diaphragm;
a first coupling member that has a pillar shape, is electrically conductively, and couples the first electrode surface of the piezoelectric element with the electrical circuit;
a second coupling member that has an annular shape, is electrically conductive, couples the second plate surface of the first diaphragm and the first plate surface of the second diaphragm, and surrounds the first coupling member in a view along the thickness direction of the second diaphragm; and
a soft member having a contact surface that is configured to contact a biological body and a non-contact surface that is configured to be away from the biological body when the contact surface is in contact with the biological body, the soft member comprising a material that is softer than the first diaphragm and the second diaphragm,
wherein:
the piezoelectric element, the first diaphragm, the second diaphragm, the first coupling member, and the second coupling member are housed in the housing,
the housing is coupled to the non-contact surface of the soft member by an adhesive member that is electrically conductive,
the second plate surface of the second diaphragm is coupled, from a center to a circumferential edge of the second plate surface, to the non-contact surface of the soft member with the adhesive member, and
the second electrode surface is electrically connected to the electrical circuit via the first diaphragm, the second coupling member, the second diaphragm, the adhesive member, and the housing.

15. A biological sound sensor comprising:
a housing;
a diaphragm having a plate shape with a first plate surface and a second plate surface on opposite sides, the diaphragm configured to vibrate in a thickness direction of the diaphragm;
a piezoelectric element on the first plate surface of the diaphragm, the piezoelectric element being configured to detect a vibration of the diaphragm; and
a soft member having a contact surface that is configured to contact a biological body and a non-contact surface that is configured to be away from the biological body when the contact surface is in contact with the biological body, the soft member being softer than the diaphragm,
wherein:
the housing has an open end that is attached to one of the first plate surface of the diaphragm or the non-contact surface of the soft member, and
the second plate surface of the diaphragm is coupled, from a center to a circumferential edge of the second plate surface, to the non-contact surface of the soft member.

16. The biological sound sensor according to claim 15, wherein the soft member has a plate shape having the contact surface and the non-contact surface on opposite sides.

17. The biological sound sensor according to claim 15, wherein the housing is totally located on an opposite side of the contact surface of the soft member across the first plate surface of the diaphragm or the non-contact surface of the soft member.

18. The biological sound sensor according to claim 15, wherein the housing is totally located behind the soft member in a thickness direction of the soft member of the diaphragm from the contact surface of the soft member.

19. The biological sound sensor according to claim 15, further comprising:
an electrical circuit that is held by the housing, wherein:
the diaphragm is electrically conductive,
the piezoelectric element has a first electrode surface and a second electrode surface, the first electrode surface being electrically connected to the electrical circuit, and
the second electrode surface is on the first plate surface of the diaphragm and is electrically connected to the electrical circuit via the diaphragm.

20. The biological sound sensor according to claim 19, wherein:
the electrical circuit and the piezoelectric element are in the housing, and
the housing is configured to hold the first plate surface of the diaphragm.
